(19) United States
(12) Patent Application Publication
(10) Pub. No.: US 2012/0259229 A1
(43) Pub. Date: Oct. 11, 2012

(54) APPARATUS AND METHODS FOR IN VIVO TISSUE CHARACTERIZATION BY RAMAN SPECTROSCOPY

(75) Inventors: Hequn Wang, Vancouver (CA); Haishan Zeng, Vancouver (CA); Harvey Lui, Vancouver (CA); Jianhua Zhao, Burnaby (CA)

(73) Assignee: BRITISH COLUMBIA CANCER AGENCY BRANCH, Vancouver, BC (CA)

(21) Appl. No.: 13/516,715

(22) PCT Filed: Dec. 17, 2010
(86) PCT No.: PCT/CA2010/001972

§ 371 Date: Jun. 15, 2012

Related U.S. Application Data
(60) Provisional application No. 61/287,500, filed on Dec. 17, 2009.

(51) Int. Cl. A61B 6/00

(52) U.S. Cl. 600/476

(57) ABSTRACT

A micro-Raman spectrometer system for use in differentiating tumor lesions from normal skin detects specific characteristics of Raman spectra indicative of cancer. A peak at 899 cm⁻¹ and a higher intensity region in the 1325 cm⁻¹ to 1330 cm⁻¹ range indicate the presence of tumors. The spectrometer system may be applied for skin cancer detection and for mapping the margins of lesions. Cancer detection methods as described herein have achieved diagnostic sensitivity of 95.8% and specificity of 93.8%.

### CROSS-REFERENCE TO RELATED APPLICATION

This application claims priority from U.S. patent application No. 61/287,500 entitled RAMAN SPECTRAL BIOMARKERS IN SKIN CANCER and filed on 17 Dec. 2009. For purposes of the United States, this application claims the benefit under 35 U.S.C. §119 of U.S. patent application No. 61/287500 filed on 17 Dec. 2009 which is hereby incorporated by reference herein.

### TECHNICAL FIELD

The invention relates to the characterization of tissues. The invention may be applied, for example, to provide methods and apparatus for assessing skin lesions. An example embodiment provides an apparatus which may be used by a physician to evaluate the likelihood that skin lesions are cancerous and to locate boundaries of such lesions.

### BACKGROUND

Skin cancer is the most common cancer in North America. One in every five North Americans are expected to develop malignant skin tumors during their lifetime. When a suspicious lesion is detected by a physician, biopsy followed by histopathologic examination is the most accurate way to confirming a diagnosis. This process is invasive, time consuming and can be associated with some morbidity. The importance of achieving high diagnostic sensitivity necessitates a low threshold for biopsy, which in turn incurs higher costs for the health care system. Furthermore a biopsy alters the site under study and leaves a permanent scar. In some cases the most appropriate site to biopsy can be difficult to ascertain.

A sensitive, specific non-invasive tool for characterizing suspicious lesions and other tissues would provide a valuable alternative to the use of biopsies and histopathologic examination of the extracted tissues.

Raman spectroscopy involves directing light at a specimen which inelastically scatters some of the incident light. Inelastic interactions with the specimen can cause the scattered light to have wavelengths that are shifted relative to the wavelength of the incident light (Raman shift). The wavelength spectrum of the scattered light (the Raman spectrum) contains information about the nature of the specimen.

The use of Raman spectroscopy in the study of tissues is described in the following references:
a) Caspers P J, et al. Raman spectroscopy in biophysics and medical physics. Biophys J 2003; 85:572-580;
b) Huang Z, et al. Rapid near-infrared Raman spectroscopy system for real-time in vivo skin measurements. Opt Lett 2001; 26:1782-1784;
c) Short M A, et al. Development and preliminary results of an endoscopic Raman probe for potential in vivo diagnosis of lung cancers. Opt Lettt 2008; 33(7):711-713;
d) Huang Z, et al. Raman spectroscopy of in vivo cutaneous melanin. J of Biomed Opt 2004; 9:1198-1205;
e) Huang Z, et al. Raman Spectroscopy in Combination with Background Near-infrared Autofluorescence Enhances the In Vivo Assessment of Malignant Tissues. Photochem Photobiol 2005; 81:1219-1226;
f) Molckovsky A, et al. Diagnostic potential of near-infrared Raman spectroscopy in the colon: differentiating adenomatous from hyperplastic polyps. Gastrointest Endosc 2003; 57:396-402;
g) Abigail S H, et al. In vivo Margin Assessment during Partial Mastectomy Breast Surgery Using Raman Spectroscopy. Cancer Res 2006; 66:3317-3322;
h) Rajadhyaksha M, et al. In Vivo Confocal Scanning Laser Microscopy of Human Skin II: Advances in Instrumentation and Comparison With Histology. J Invest Dermatol 1999; 113:293-303;
i) Lieber C A, et al. In vivo nonmelanoma skin cancer diagnosis using Raman microspectroscopy. Laser Surg Med 2008; 40(7):461-467.
   All of these references are hereby incorporated herein by reference.

The use of optical apparatus which applies Raman spectroscopy to analyze light collected using confocal techniques is described in
j) Caspers P J, et al. Automated depth-scanning confocal Raman microspectrometer for rapid in vivo determination of water concentration profiles in human skin. J Raman Spectrosc 2000; 31:813-818;
k) Caspers P J,et al. In vivo confocal Raman microspectroscopy of the skin: noninvasive determination of molecular concentration profiles. J Invest Dermatol 2001; 116:434-442;
l) Caspers P J, et al. Monitoring the penetration enhancer dimethyl sulfoxide in human stratum corneum in vivo by confocal Raman spectroscopy. Pharm Res 2002; 19:1577-1580.
   All of these references are hereby incorporated herein by reference.

There is a need for sensitive and specific methods for screening for skin cancers such as melanomas. There is also a need for tools which can be used by physicians to accurately detect the margins of lesions.

### SUMMARY OF THE INVENTION

This invention has a number of aspects. These aspects include: apparatus useful for assessing the pathology of tissue (e.g. skin) in vivo; methods useful for assessing the pathology of tissue (e.g. skin) in vivo; apparatus for processing tissue Raman spectroscopy data and generating a measure of the likelihood that the spectra correspond to cancerous or pre-cancerous tissues; methods for processing tissue Raman spectroscopy data and generating a measure of the likelihood that the spectra correspond to cancerous or pre-cancerous tissues; non-transitory media containing computer-readable instructions that, when executed by a data processor cause the data processor to execute a method for processing tissue Raman spectroscopy data and generating a measure of the likelihood that the spectra correspond to cancerous or pre- cancerous tissues.

One aspect of the invention provides an apparatus for tissue characterization comprising a confocal Raman spectrometer configured to generate a Raman spectrum, a Raman spectrum analysis unit configured to measure at least one characteristic of the Raman spectrum, and an indicator device driven in response to the measured characteristic. The at least one characteristic including one or more of a first characteristic that relates to a peak at a wavenumber of 899±10 cm⁻¹ and a second characteristic that relates to a comparison of the intensity of the Raman spectrum in a first range within a wavenumber band from 1240±10 cm⁻¹ to 1269±10 cm⁻¹ to the intensity in a second range within a wavenumber band from 1269±10 cm⁻¹ to 1340±10 cm⁻¹.

Another aspect of the invention provides a method for tissue characterization involving receiving at least one Raman spectrum of a tissue, measuring at least one characteristic of the Raman spectrum, characterizing the tissue in response to the measured characteristic, and generating an indication of the characterization of the tissue. The characteristic comprising at least one of a first characteristic that relates to a magnitude of the intensity of the Raman spectrum at a wavenumber of 899±10 cm⁻¹, and a second characteristic that relates to a comparison of the intensity of the Raman spectrum in a first range within a wavenumber band from 1240±10 cm⁻¹ to 1269±10 cm⁻¹ to the intensity in a second range within a wavenumber band from 1269±10 cm⁻¹ to 1340±10 cm⁻¹.

Another aspect of the invention provides a non-transitory tangible computer-readable medium storing instructions for execution by at least one data-processor that, when executed by the data-processor cause the data processor to execute a method for characterizing tissue comprising the steps of processing at least one Raman spectrum of a tissue, measuring at least one characteristic of the Raman spectrum, characterizing the tissue in response to the measured at least one characteristic, and generating an indication of the characterization of the tissue. The at least one characteristic comprises one or more of a first characteristic that relates to a magnitude of the intensity of the Raman spectrum at a wavenumber of 899±10 cm⁻¹, and a second characteristic that relates to a comparison of the intensity of the Raman spectrum in a first range within a wavenumber band from 1240±10 cm⁻¹ to 1269±10 cm⁻¹ to the intensity in a second range within a wavenumber hand from 1269±10 cm⁻¹ to 1340±10 cm⁻¹.

Additional aspects of the invention and features of example embodiments of the invention are described in the following description and/or illustrated in the accompanying drawings.

### BRIEF DESCRIPTION OF THE DRAWINGS

The accompanying drawings illustrate non-limiting embodiments of the invention.

FIG. 1 is a block diagram of a diagnostic apparatus according to an example embodiment of the invention.

FIG. 2 is a block diagram of an apparatus according to another example embodiment of the invention.

FIG. 3A is a graph of a raw Raman spectrum.

FIG. 3B is a graph of the Raman spectrum of FIG. 3A with a polynomial curve fit to the fluorescence background.

FIG. 3C is a graph of the Raman spectrum of FIG. 3A with the fluorescence background subtracted.

FIG. 4 is a graph of an example Raman spectra at the epidermal layer.

FIG. 4A is an expanded view of the graph of FIG. 4.

FIG. 5 is a graph of an example Raman spectra at the dermal layer.

FIG. 6 is a block diagram of a method according to an example embodiment of the invention.

FIG. 7 is a scatter plot of example Principal Component (PC) scores for dermal spectra.

FIG. 8 is a graph of an example receiver operating characteristic (ROC) curve for dermal spectra.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

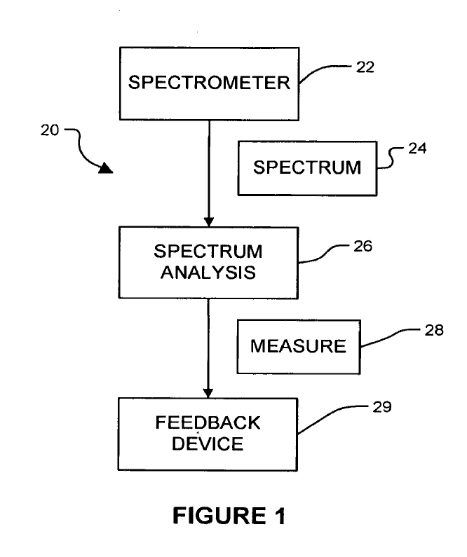

FIG. 1 is a block diagram of apparatus 20 according to an example embodiment of the invention. Apparatus 20 comprises a Raman spectrometer 22 which is configured to determine a Raman spectrum 24 for a small volume of a tissue T. Tissue T may be skin, for example.

A spectrum analysis component 26 receives Raman spectrum 24 and processes the Raman spectrum to obtain a measure 28 indicative of the pathology of the tissue for which Raman spectrum 24 was obtained. Measure 28 controls a feedback device 29. Feedback device 29 may, for example, comprise a lamp, graphical indication, sound, display or other device which provides a human-perceptible signal in response to measure 28.

Measure 28 is based at least in part upon one or both of two specific features of Raman spectrum 24. These features are a peak at a Raman shift of 899 cm⁻¹ and relative intensities in the ranges of approximately 1240 cm⁻¹ to 1269 cm⁻¹ and 1269 cm⁻¹ to 1340 cm⁻¹. The second feature may, for example, comprise a ratio of the integrated intensity in the range of 1240 cm⁻¹ to 1269 cm⁻¹ to the integrated intensity in the range of 1269 cm⁻¹ to 1340 cm⁻¹. The endpoints of these ranges may be varied somewhat e.g. by ±10 cm⁻¹ or ±2 cm^{—1} while still providing a comparison that has diagnostic value.

In some embodiments, spectrometer 22 is of a type that can be controlled to selectively acquire Raman spectra from tissues at different depths. In some embodiments, Raman spectrometer 22 is controllable to acquire (in any order) a first Raman spectrum corresponding to the epidermis (e.g. a spectrum relating to tissues at a depth of 0 to about 25 μm) and a second Raman spectrum relating to the dermis (e.g. a spectrum relating to tissues at a depth greater than 25 μm). In some embodiments spectrum analysis component 26 performs different analysis of a Raman spectrum corresponding to the epidermis and a Raman spectrum corresponding to the dermis.

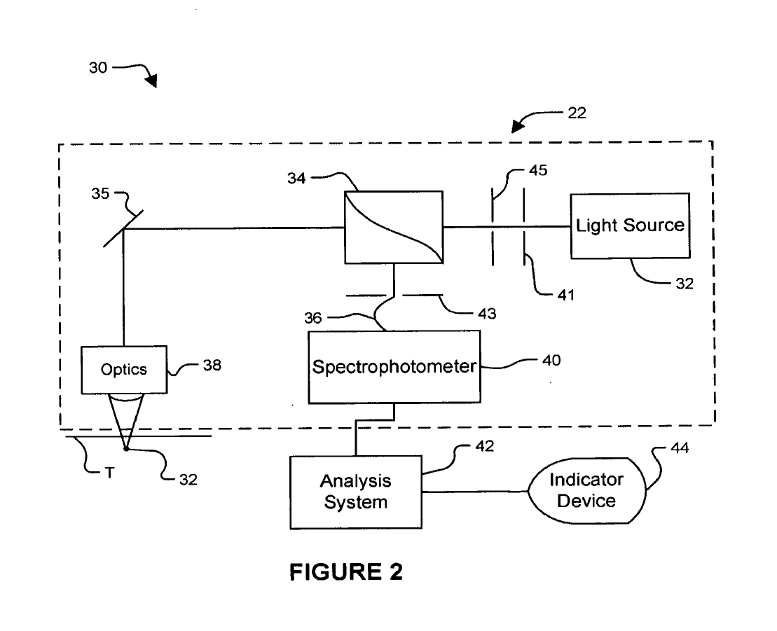

FIG. 2 is a block diagram of apparatus 30 according to another example embodiment of the invention. In FIG. 2, Raman spectrometer 22 is shown to comprise a light source 32. Light source 32 is a monochromatic light source and may, for example, comprise a laser. Light source 32 may, for example, comprise a single-mode stabilized diode laser operating at a wavelength of 785 nm and having a power of 100 mW. In a prototype embodiment; the light source was a Model 10785SU0100B-TK laser from Innovative Photonic Solutions of Monmouth Junction, NJ.

In apparatus 30, light from light source 32 is collected, passed through a band-pass filter 45 and beam splitter 34 and directed via mirror 35 to optics 38 which focus the light to a spot 39 within the tissue T being studied. Tissue T may comprise an area of the skin of a person or animal for example. In the prototype embodiment, waveguide 36 comprised a 100 μm core-diameter low-OH single fiber, which had a high near-infrared (NIR) transmission.

In the prototype embodiment, optics 38 comprised a water-immersion objective lens (specifically an Olympus™ Model No. LUMPL40 W/IR, N.A. 0.8, WD 3.3 mm objective lens). A magnetic adapter ring (item #02934, available from Lucid, Inc. Rochester, N.Y.) was affixed to the area of interest with double-sided adheive tape. The adapter ring held optics 38 in position relative to the tissues being studied.

Light scattered by tissue at focus spot 39 is collected by optics 38 and passed through beam splitter 34, a long-pass filter 43 and into waveguide 36 (such as an optical fiber) to be transmitted to spectrophotometer 40. In the prototype embodiment, waveguide 36 comprised a 100 μm core-diameter low-OH single fiber, which had a high near-infrared (NIR) transmission.

In the prototype embodiment, optics 38 comprised a water-immersion objective lens (specifically an Olympus™ Model No. LUMPL40 W/IR, N.A. 0.8, WD 3.3 mm objective lens). A magnetic adapter ring (item #02934, available from Lucid, Inc. Rochester, N.Y.) was affixed to the area of interest with double-sided adhesive tape. The adapter ring held optics 38 in position relative to the tissues being studied.

It is desirable to avoid exposing tissues to excessive amounts of radiation. This may he achieved by appropriate selection of light source, control of the light source, and/or providing attenuation downstream from the light source. In the prototype embodiment the light intensity after optics 38 and incident on the tissue surface was 27 mW.

Spectrophotometer 40 measures a spectrum of the light. In the prototype embodiment, spectrophotometer 40 comprised a NIR-optimized back illumination deep-depletion charge-coupled device (CCD) array and a transmissive imaging spectrograph with a volume phase technology holographic grating. The CCD had a 16 bit dynamic range and was cooled with liquid nitrogen to −120° C. In the prototype the CCD was a model Spec-10:100BR/LN from Princeton Instruments, Trenton, N.J. and the spectrometer comprised a HoloSpec™-f/2.2-NIR, spectrometer from Kaiser Optical Systems Inc. of Ann Arbor, Mich. with a volume phase technology holographic grating model HSG-785-LF from Kaiser Optical Systems Inc., Ann Arbor, Mich.

In a preferred embodiment, Raman spectrometer 22 comprises a confocal optical arrangement wherein the light source comprises a point source of light and a spatial pinhole or other spatial filter 41 is provided to block out-of-focus light from reaching the spectrophotometer 40. This permits Raman spectra to be obtained for points at specific depths within tissue T. This capability is exploited in some embodiments to obtain separate Raman spectra for epidermal and dermal tissues at the same location.

The spectral resolution of the prototype system was 8 cm⁻¹. The axial (depth) resolution and lateral resolution of the prototype system were measured to be 8.6 μm and 2.2 μm, respectively. The spectrophotometer was able to acquire spectra over the wavenumber range of 800-1800 cm⁻¹ (equivalent to a wavelength range of 838-914 nm). Raman spectra of skin tissues with good signal-to-noise ratio (SNR) were obtained within 15 seconds with an exposure level of 27 mW at the skin surface.

A spectrum analysis system 42 analyzes spectra from spectrophotometer 40. Spectrum analysis system 42 is configured to identify specific spectral characteristics of Raman spectra received from spectrophotometer 40.

Spectrum analysis system 42 may comprise a programmed data processor such as a personal computer, an embedded computer, a microprocessor, a graphics processor, a digital signal processor or the like executing software and/or firmware instructions that cause the processor to extract the specific spectral characteristics from the Raman spectra. In alternative embodiments spectrum analysis system 42 comprises electronic circuits, logic pipelines or other hardware that is configured to extract the specific spectral characteristics or a programmed data processor in combination with hardware that performs one or more steps in the extraction of the specific spectral characteristics.

It is convenient but not mandatory for spectrum analysis system 42 to operate in real time or near real time such that analysis of a Raman spectrum is completed at essentially the same time or at least within a few seconds of the Raman spectrum being acquired.

Spectrum analysis system 42 is connected to control an indicator device 44 according to a measure derived from the specific spectral characteristics extracted from the Raman spectrum by spectrum analysis unit 42.

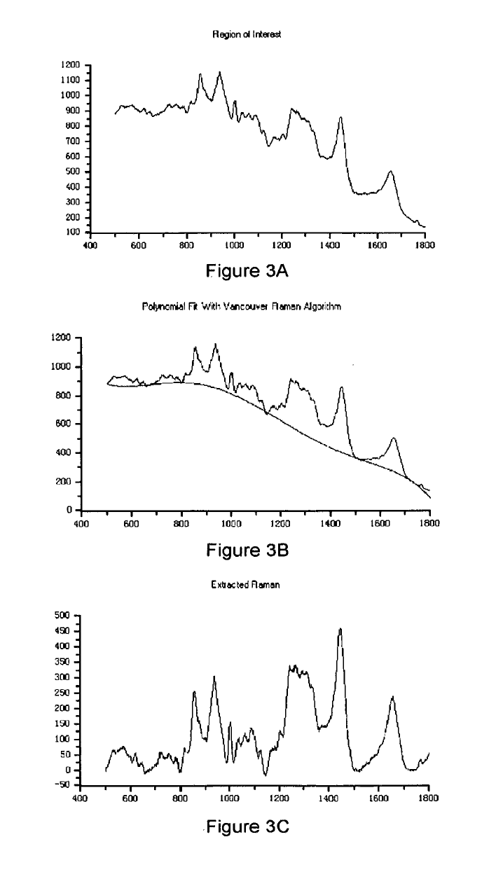

The measured Raman spectra are typically superimposed on a fluorescence background, which varies with each measurement. It is convenient for spectrum analysis system 42 to process received spectra to remove the fluorescence background and also to normalize the spectra. Removal of fluorescence background may be achieved, for example using the Vancouver Raman Algorithm as described in Zhao J, et al. Automated Autofluorescence Background Subtraction Algorithm for Biomedical Raman Spectroscopy. Appl. Spectrosc. 2007; 61:1225-1232, which is hereby incorporated herein by reference. The Vancouver Raman Algorithm is an iterative modified polynomial curve fitting fluorescence removal method that takes noise into account. FIGS. 3A, 3B and 3C respectively show a raw Raman spectrum, the Raman spectrum of FIG. 3A with a polynomial curve fit to the fluorescence background and the Raman spectrum of FIG. 3A with the fluorescence background as modeled by the polynomial curve subtracted.

Normalization may be performed, for example, to the area under curve (AUC) of each spectrum. For example, each spectrum may be multiplied by a value selected to make the AUC equal to a standard value. For convenience in displaying the spectra, the normalized intensities may be divided by the number of data points in each spectrum.

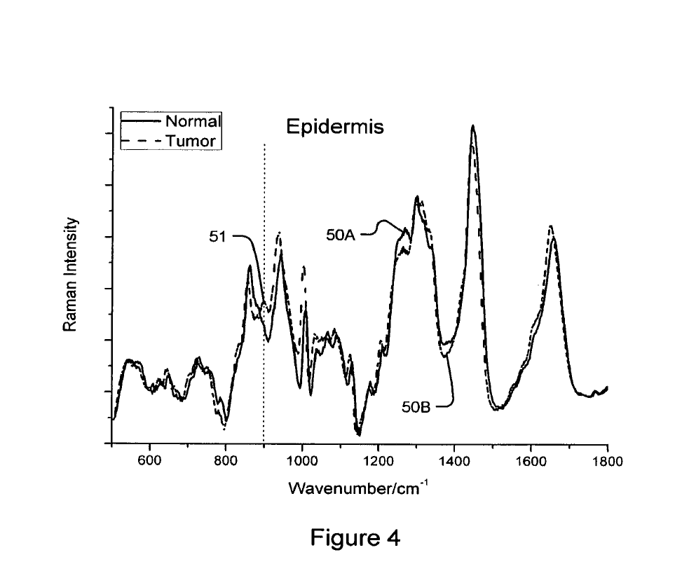

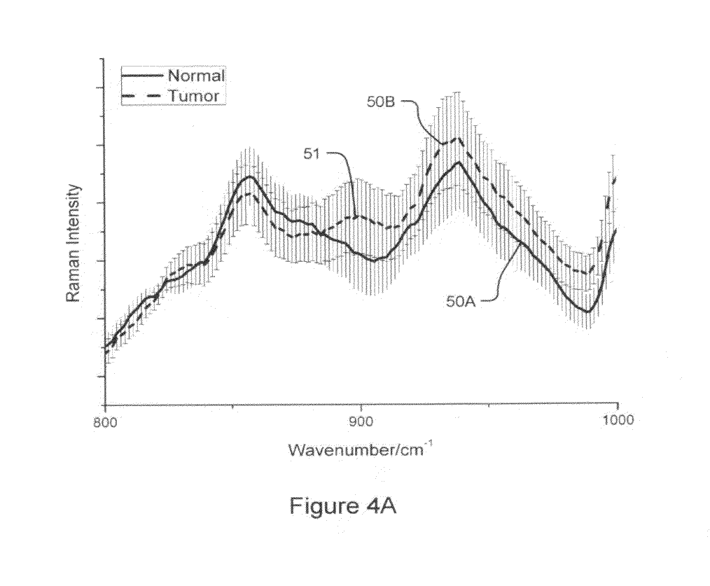

FIG. 4 shows example Raman spectra at the epidermal level for normal skin (curve 50A) and for a tumor (curve 50B). This Figure illustrates a first specific spectral characteristic that may be extracted by spectrum analysis unit 42. The first spectral characteristic is the peak 51 at a wavenumber of approximately 899 cm⁻¹ that is present in tumor spectrum 50B and not present in normal spectrum 50B. Peak 51 is also shown in FIG. 4A which is an expanded view of the portions of spectra 50A and 50B in the wavenumber range of 800 cm⁻¹ to 1000 cm⁻¹. Thus, detecting the peak at 899 cm⁻¹ in epidermal tissues is one way to evaluate whether the tissue is normal or tumor tissue.

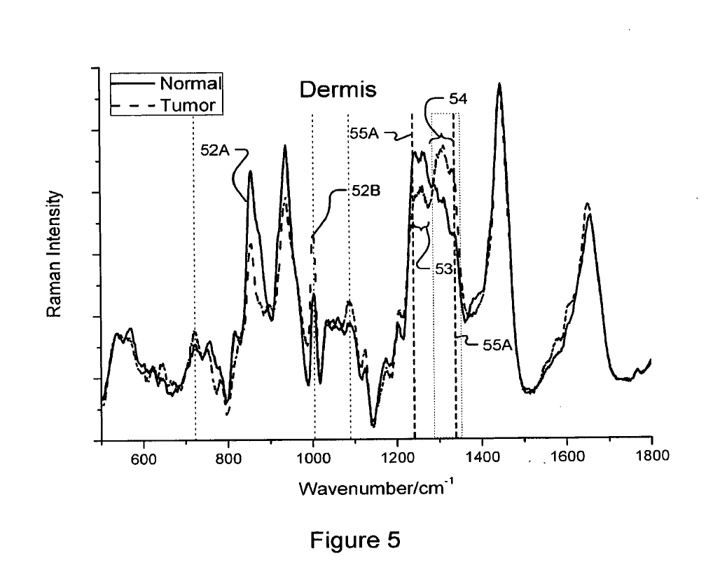

A second spectral characteristic that may be extracted from Raman spectra by spectrum analysis unit 42 is illustrated in FIG. 5 which shows example Raman spectra at the dermis level for normal skin (curve 52A) and for a tumor (curve 52B). It can be seen that in a wavenumber range 53 from about 1240 cm⁻¹ to 1269 cm⁻¹ normal spectrum 52A is greater than tumor spectrum 52B while in a nearby wavenumber range 54 from about 1269 cm⁻¹ to 1340 cm^{—1} normal spectrum 52A is less than tumor spectrum 52B. Comparison of the spectra in ranges 53 and 54 therefore provides a second spectral characteristic that characterizes the tissue either on its own or in addition to the first spectral characteristic.

Comparison may be performed, for example, by computing a ratio of spectrum intensities at selected wavenumbers within ranges 53 and 54 or a ratio of the integrated intensity in range 53 to that in range 54. These ratios will tend to be larger than unity for normal tissue and less than unity for tumor tissue. Thus, comparing the ratio of the integrated intensity to a threshold is one way to evaluate whether the tissue is normal or tumor tissue.

Another way to compare the spectra in ranges 53 and 54 is.to fit a line to points on the spectral curve in a region that includes all or part of ranges 53 and 54. For example, a line may be fit to the portion of the spectral curve between points 55A and 55B. In the illustrated embodiment, points 55A and 55B correspond respectively to wavenumbers of 1240 cm⁻¹ and 1340 cm⁻¹. A negative slope, or negative differential between intensities, corresponds to normal tissue and a positive slope, or positive differential between intensities, corresponds to tumor tissue. In another example, a line may be fit to the portion of the spectral curve between points of maximum intensity in ranges 53 and 54. Again, a negative slope corresponds to normal tissue and a positive slope corresponds to tumor tissue.

Another approach is to measure the peaks in ranges within the 1240-1269 cm⁻¹ range and the 1269-1340 cm⁻¹ range. For example, peaks may be measured in one or both of the 1325 to 1330 cm⁻¹ range and the 1222 to 1266 cm⁻¹ range. The measured peak(s) may be compared to thresholds for the purpose of evaluating the likelihood that the spectrum corresponds to abnormal tissue.

Various different techniques may be applied to analyzing Raman spectra to determine measures of the specific spectral characteristics indicative of tumor tissue. For example, a suitable peak finding and measurement function may be applied to measure the peak at 899 cm⁻¹ and/or the peaks in the 1325 to 1330 cm⁻¹ range and the 1222 to 1266 cm⁻¹ range. A wide range of such peak measurement functions are known to those of skill in the art. Various suitable peak finding and measurement algorithms are commercially available.

Another approach to generating measures of the specific spectral characteristics is to apply multivariate data analysis. For example, a particular spectrum may be analyzed by performing a principle component analysis (PCA). PCA may be performed on part or all of the range of the acquired Raman spectra (e.g. 500 cm⁻¹ to 1800 cm⁻¹).

PCA involves generating a set of principle components which represent a given proportion of the variance in a set of training spectra. For example, in the prototype embodiment, each spectrum of epidermal tissue was represented as a linear combination of a set of 4 PCA variables and each spectrum of dermal tissue was represented as a linear combination of a set of 3 PCA variables. In each case the PCA variables represented at least 70% of the total variance of the set of training spectra.

Principal components (PCs) may be derived by performing PCA on the standardized spectral data matrix to generate PCs. The PCs generally provide a reduced number of orthogonal variables that account for most of the total variance in original spectra. Where the training set of Raman spectra includes both Raman spectra of tumor tissue in which the first and second characteristics are present and Raman spectra of normal tissue in which the first and second characteristics are not present, the first and second characteristics will contribute significantly to the total variance in the spectra of the training set. Therefore, PCs generated with such a training set provide another mechanism for extracting the first and second characteristics from the Raman spectra.

PCs may be used to assess a new Raman spectrum by computing a variable called the PC score, which represents the weight of that particular component in the Raman spectrum being analyzed.

Linear discriminant analysis (LDA) can then be used to derive a function of the PC scores which indicates whether or not the tissue is normal. In the prototype embodiment. for analysis of Raman spectra for tissues in the dermis, the first three PC scores which have the largest eigenvalues were used for tissue classification. For analysis of Raman spectra of tissue of the epidermis the first four PC scores were used. LDA was applied to determine a discriminate function line that maximized the variance in the data between groups (e.g. “normal” and “tumor” groups) while minimizing the variance between members of the same group.

The discriminate function line may subsequently be applied to categorize an unknown tissue based on where a point corresponding to the PC scores for a Raman spectrum of the unknown tissue is relative to the discriminate function line.

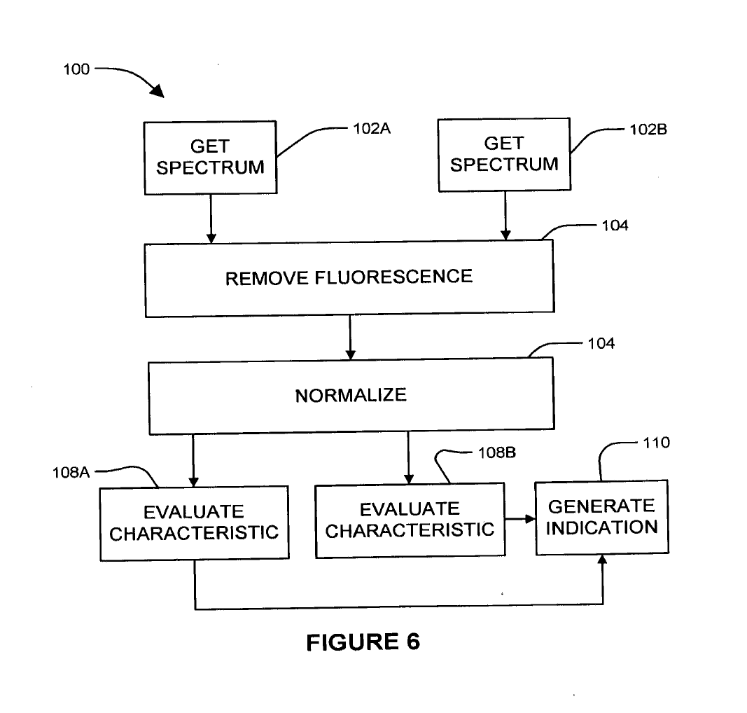

FIG. 6 illustrates a method 100 according to an example embodiment of the invention. Method 100 operates a Raman spectrometer to obtain a first Raman spectrum of a subject's tissue at a first depth in block 102A and to obtain a second Raman spectrum of the subject's tissue at a second depth in block 102B. In some embodiments the first depth corresponds to epidermal tissue (e.g. is a depth in the range of 0 to 25 μm) and the second depth corresponds to dermal tissue (e.g. is a depth in excess of 25 μm such as a depth in the range of 25 to 50 μm). Blocks 102A and 102B may be performed with a probe that is held in the same position against a living subject.

In block 104 the fluorescent background is removed from the Raman spectra. In block 105 the Raman spectra are normalized.

In block 108A the first Raman spectrum is processed to evaluate a first characteristic. For example, the first Raman spectrum may be processed to evaluate the degree to which it includes a peak in the vicinity of 899 cm⁻¹. In block 108B the second Raman spectrum is processed to evaluate a second characteristic. For example, the second Raman spectrum may be processed to obtain a measure of the degree to which the second spectrum is more intense in the region of 1240 cm⁻¹ to 1269 cm⁻¹ than it is in the region of 1269 cm⁻¹ to 1340 cm^{—1}.

In block 110 an indication is displayed. The indication is based on the outputs of one or both of blocks 108A and 108B.

Simpler versions of method 100 leave out blocks 102A and 108A or leave out blocks 102B and 108B.

It is not mandatory to obtain a complete high signal-to-noise ratio Raman spectrum for every point or at every depth. If enough Raman spectrum information has been collected for a point for it to be sure that the indication will be positive for that point (e.g. there is enough information to determine that a peak at 899 cm^{—1} is present clearly enough to support a diagnosis of cancer - a positive indication) then data collection for that point may be stopped. If the Raman spectrum of block 102A clearly supports a positive indication for a point then the method may skip block 102B and associated processing steps.

### Example Application

A dermatologist has a patient who has a suspicious-looking lesion. The dermatologist has apparatus as described herein. The dermatologist places the probe against the lesion and acquires one or more Raman spectra for tissue in the lesion. The apparatus detects one or more of the specific spectral characteristics as described herein and, in response to detecting the spectral characteristics provides an indication to the dermatologist that the lesion is not normal. For example, the apparatus may include a signal light that indicates green for normal tissue (lack of spectral characteristics indicating tumor tissue) and red for tumor tissue (one or more spectral characteristics are indicative of abnormal tissue pathology consistent with a cancerous tumor and/or a pre-cancerous lesion).

The dermatologist decides to take a biopsy and to send a sample from the biopsy for histopathologic examination. If the apparatus had indicated normal tissue and a visual examination of the lesion was inconclusive the dermatologist might not have ordered a biopsy.

The biopsy results confirm that the lesion is cancerous and must be excised. The dermatologist uses the apparatus to locate margins of the lesion by marking the points nearest to the lesion where the apparatus indicates that the tissue is normal. The dermatologist then operates to remove the lesion. Because the margins of the lesion have been identified the entire lesion can be removed without removing excess tissue.

In some embodiments the apparatus comprises a hand-held probe that includes a skin marking device and the dermatologist operates the skin marking device to mark on the subject's skin points where Raman spectra have been acquired. In some embodiments the marking is different depending on the indication for the point.

### Experimental Validation

494 Raman spectra were taken in vivo from 24 tumor bearing mice in order to assess: (1) the Raman spectral differences between different skin layers and (2) the spectral changes for both the epidermis and the dermis between normal peritumoral skin and skin immediately overlying subcutaneous tumors.

All animal experiments were performed according to a protocol approved by the University of British Columbia Committee on Animal Care. The squamous cell carcinoma (SCCVII) tumors were generated by subcutaneous injection of 3.6×10⁶ cells in 50 μL phosphate buffered saline (PBS) into the back of female C3H/HeN mice. Raman spectroscopy was performed when the tumor volume reached 90 to 120 mm³ (˜10 days after tumor inoculation). The dimensions of each tumor were measured by a caliper every other day and their volumes were calculated by volume=(π/6)×(tumor length)×(tumor width)×(tumor height). All mice were shaved and anesthetized before measurement. Axial scanning from the skin surface to deeper layers was performed both at the tumor site and a normal-appearing skin site (approximately 3-4 cm away from the tumor site) within the same anatomic region.

After each experiment, the skin under measurement was excised, processed for histologic examination, and the skin sections stained with hematoxylin and eosin (H&E). 264 spectra from normal sites and 230 spectra from tumor sites at depths ranging from 10 μm to 140 μm below the skin surface were acquired.

PCA was performed on the resulting spectra. Four sets including 48 normal spectra (10 μm and 20 μm depth), 48 tumor spectra (10 μm and 20 μm depths), 48 normal spectra (30 μm and 40 μm depths), and 48 tumor spectra (30 μm and 40 μm depths) were used in the PCA.

For the epidermis (10 μm and 20 μm depths) four PCs retaining 70% of the variance of the original data were kept for discriminate analysis to differentiate the tumor from normal. For the dermis (30 μm and 40 μm depths) three PCs accounted for 70% of the variance and were used for analysis.

Leave-one-out cross validation procedures were used in order to prevent over training. In this method, one spectrum was removed from the data set and the entire algorithm, including PCA and LDA, was redeveloped and optimized using the remaining spectral set. The optimized algorithm was then used to classify the withheld spectrum and this process was repeated until each spectrum was individually classified.

The three PCs for dermis are plotted in FIG. 6 which shows that the PCs picked up the information coming from collagen (855 cm⁻¹ and 937 cm⁻¹), phenylalanine (1001 cm⁻¹), lipids (1061 cm⁻¹, 1128 cm⁻¹, 1296 cm⁻¹), and nucleic acids (1325-1330 cm⁻¹). This is in good correlation with the major differences observable in the spectra between normal and tumor groups in the dermis.

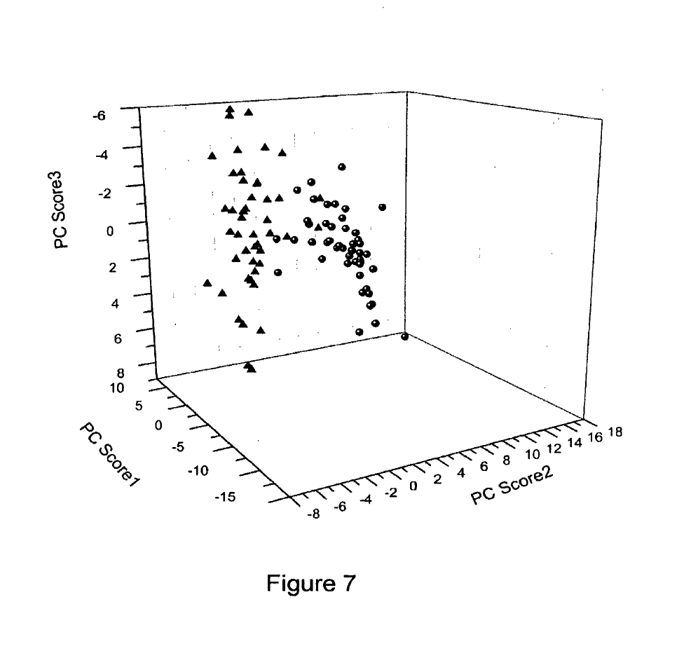

In the epidermis, the PCs also picked up the 899 cm⁻¹ signal which is the most significant difference between normal and tumor-bearing skin. FIG. 7 is a scatter plot of the three PC scores (PC 1, 2, and 3) for the dermal spectra, demonstrating that the two groups (normal skin vs. tumor) can be very well separated. Analysis of the PCs provided an optimal diagnostic sensitivity of 95.8% and specificity of 93.8%.

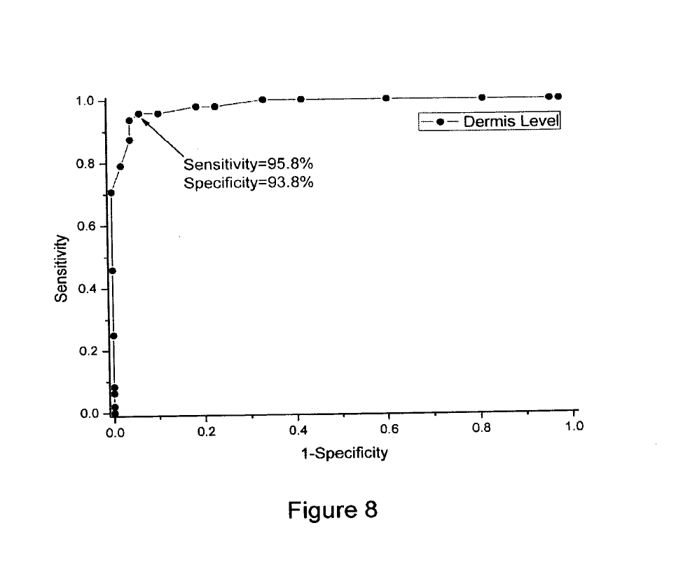

To evaluate the performance of the PCA-LDA model for tissue classification using the spectroscopic data set, receiver operating characteristic (ROC) curves were generated by successively changing the thresholds to determine correct and incorrect classification for all samples. All multivariate statistical analyses were performed using MatLab™ software (Version 7.6, MatLab™ Software, the MathWorks Inc., Mass.) with the Statistical Pattern Recognition Toolbox (Vojtech Franc and Vaclav Hlavac, Czech Technical University Prague, Faculty of Electrical Engineering, Center for Machine Perception, Czech Republic). The area under the ROC curve was 0.96 (see FIG. 8).

For the epidermal spectra, an optimal sensitivity of 89.6%, specificity of 89.6% and AUC of 0.88 were obtained.

As an illustration of another approach to tissue classification using the specific spectral features described above the peak at 899 cm⁻¹ was identified by visual inspection and used to sort spectra at the epidermis level into two groups. Two normal spectra showed this peak (providing ‘false positives’) and 2 tumor spectra did not include this peak. The overall sensitivity was 95.8% and the specificity was 95.8%.

As another illustration the ratio (R) of the integrated intensity from 1240 cm⁻¹ to 1269 cm⁻¹ to the integrated intensity from 1269 cm⁻¹ to 1340 cm⁻¹ was calculated for the spectra at dermis level. 9 normal spectra showed a ratio smaller than one (indicating that higher concentrations of nucleic acids were present) whereas 2 tumor cases showed a ratio larger than one (indicating that lower concentrations of nucleic acids were present). This measure provided a sensitivity of 95.8% and a specificity of 81.3%.

A diagnostic test which indicates cancer if either the first or second characteristic of the Raman spectrum is present was found to have a sensitivity of 100% and a specificity of 79.2%.

Certain implementations of the invention comprise computer processors which execute software instructions which cause the processors to perform a method of the invention. For example, one or more processors in a medical Raman specrometer may implement methods as described herein by executing software instructions in a program memory accessible to the processors. The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like or transmission-type media such as digital or analog communication links. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component, any component which performs the function of the described component (i.e., that is functionally equivalent), including components which are not structurally equivalent to the disclosed structure which perform the function in the illustrated exemplary embodiments of the invention.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. Apparatus for tissue characterization, the apparatus comprising:
a confocal Raman spectrometer configured to generate a Raman spectrum;
a Raman spectrum analysis unit configured to determine at least one characteristic of the Raman spectrum, the at least one characteristic including one or more of:
a first characteristic based on a magnitude of a peak at a wavenumber of 899±10 cm⁻¹; and
a second characteristic based on comparison of the intensity of the Raman spectrum in a first range within a wavenumber band from 1240±10 cm⁻¹ to 1269±10 cm⁻¹ to the intensity in a second range within a wavenumber band from 1269±10 cm⁻¹ to 1340±10 cm⁻¹; and
an indicator device driven in response to the at least one characteristic.

2. Apparatus according to claim 1 wherein the confocal Raman spectrometer has a variable depth of focus and is configured to obtain a first Raman spectrum at a first depth of focus corresponding to epidermal tissues and a second Raman spectrum at a second depth of focus corresponding to dermal tissues.

3. Apparatus according to claim 2 wherein the Raman spectrum analysis unit is configured to measure the first characteristic for the first Raman spectrum and to measure the second characteristic for the second Raman spectrum.

4. Apparatus according to claim 1 wherein the second characteristic comprises a ratio of the integrated intensity in the first range and the integrated intensity in the second range.

5. Apparatus according to claim 4 wherein the first range is 1240±2 cm⁻¹ to 1269±2 cm⁻¹ and the second range is 1269±2 cm⁻¹ to 1340±2 cm^{—1}.

6. Apparatus according to claim 1 wherein the indicator device comprises a lamp.

7. Apparatus according to claim 1 wherein the confocal Raman spectrometer comprises a hand-held probe.

8. Apparatus according to claim 1 wherein the Raman spectrum analysis unit comprises a fluorescence background subtraction stage configured to subtract a fluorescence background from the Raman spectrum.

9. Apparatus according to claim 8 wherein the Raman spectrum analysis unit comprises a normalization stage following the fluorescence background subtraction stage, the normalization stage configured to normalize the Raman spectrum.

10. Apparatus according to claim 1 wherein the indicator device is configured to mark a surface of the tissue in response to the measured at least one characteristic.

11. Apparatus according to claim 1 wherein the Raman spectrum analysis unit comprises a characterization stage configured to characterize the tissue as normal or abnormal in response to the measured at least one characteristic.

12. Apparatus according to claim 11 wherein the indicator device is configured to generate an outline of abnormal tissue.

13. A method for tissue characterization comprising:
obtaining at least one Raman spectrum of a tissue;
in a programmed spectrum analysis unit comprising a data processor executing software instructions, determining at least one characteristic of the
a first characteristic based on a magnitude of the intensity of the Raman spectrum at a wavenumber of 899±10 cm⁻¹; and
a second characteristic based on a comparison of the intensity of the Raman spectrum in a first range within a wavenumber band from 1240±10 cm⁻¹ to 1269±10 cm⁻¹ to the intensity in a second range within a wavenumber band from 1269±10 cm⁻¹ to 1340±10 cm⁻¹; and
generating an indication in response to the measured at least one characteristic.

14. A method according to claim 13 further comprising acquiring the Raman spectrum with a confocal Raman spectrometer.

15. A method according to claim 13 comprising performing a fluorescence background subtraction step to remove a fluorescence background from the Raman spectrum prior to determining the at least one characteristic.

16. A method according to claim 15 comprising normalizing the Raman spectrum following the fluorescence background subtraction step.

17. A method according to claim 13 wherein the Raman spectrum comprises a first Raman spectrum corresponding to epidermal tissues and a second Raman spectrum corresponding to dermal tissues and the method comprises separately determining the at least one characteristic for each of the first and second Raman spectra.

18. A method according to claim 13 wherein determining the at least one characteristic comprises one or more of:
in a first comparison comparing the first characteristic to a first threshold value and characterizing the tissue as abnormal based on a result of the first comparison; and
in a second comparison comparing the second characteristic to a second threshold value and characterizing the tissue as abnormal based on a result of the second comparison;.

19. A method according to claim 13 wherein the second characteristic comprises a ratio of the integrated intensity in the first range and the integrated intensity in the second range.

20. A method according to claim 13 wherein determining the second characteristic comprises comparing a maximum intensity of the Raman spectrum in the first range to a maximum intensityof the Raman spectrum in the second range.

21. A method according to claim 13 wherein the second characteristic comprises, a comparison between:
a ratio of the intensity of the Raman spectrum within the first range and a standard intensity within the first range; and
a ratio of the intensity of the Raman spectrum in the second range and a standard intensity within the second range.

22. A method according to claim 13 wherein the second characteristic comprises a slope of a line between a point of maximum intensity of the Raman spectrum within the first range and a point of maximum intensity of the Raman spectrum within the second range.

23. A method according to claim 22 comprising characterizing the tissue as normal if the slope of the line is negative and characterizing the tissue as abnormal if the slope of the line is positive.

24. A method according to claim 13 wherein the second characteristic is a slope of a line between an intensity of the Raman spectrum at a wavenumber of 1240 cm⁻¹ and an intensity of the Raman spectrum at a wavenumber of 1340 cm⁻¹.

25. A method according to claim 24 comprising the step of characterizing the tissue as normal if the slope of the line is negative and characterizing the tissue as abnormal if the slope of the line is positive.

26. A method according to claim 13 comprising the step of generating a likelihood that the tissue is abnormal corresponding to a predetermined sensitivity of the at least one characteristic.

27. A method according to claim 26 wherein the step of generating an indication comprises generating an indication of the likelihood that the tissue is abnormal.

28. A method according to claim 13 wherein the step of generating an indication comprises generating an outline for a surface of the tissue in response to the measured at least one characteristic.

29. A method according to claim 13 wherein the step generating an indication comprises marking the tissue surface.

30. A method according to claim 29 wherein the outline is marked on the tissue surface by the confocal Raman spectrometer.

31. A non-transitory tangible computer-readable medium storing instructions for execution by at least one data-processor that, when executed by the data-processor cause the data processor to execute a method for characterizing tissue comprising the steps of:
receiving at least one Raman spectrum of a tissue;
measuring at least one characteristic of the Raman spectrum, the at least one characteristic comprising one or more of:
a first characteristic based on a magnitude of the intensity of the Raman spectrum at a wavenumber of 899±10 cm⁻¹; and
a second characteristic based on a comparison of the intensity of the Raman spectrum in a first range within a wavenumber band from 1240±10 cm⁻¹ to 1269±10 cm^{—1} to the intensity in a second range within a wavenumber band from 1269±10 cm⁻¹ to 1340±10 cm⁻¹;
characterizing the tissue in response to the measured at least one characteristic; and
generating an indication of the characterization of the tissue.

32. The non-transitory tangible computer-readable medium of claim 31, wherein the non-transitory tangible computer-readable medium further stores the at least one Raman spectrum.
